(19) United States
(12) Patent Application Publication
(10) Pub. No.: US 2018/0160954 A1
(43) Pub. Date: Jun. 14, 2018

(54) PULSE OXIMETER

(71) Applicant: HON HAI PRECISION INDUSTRY CO., LTD., New Taipei (TW)

(72) Inventors: JUIN-HONG LIN, New Taipei (TW); CHAO-TSANG WEI, New Taipei (TW); HO-CHIANG LIU, New Taipei (TW)

(21) Appl. No.: 15/686,589

(22) Filed: Aug. 25, 2017

(30) Foreign Application Priority Data
Dec. 14, 2016 (TW) 105141480

(51) Int. Cl. A61B 5/1455; A61B 5/0205; A61B 5/00; G01J 3/12

(52) U.S. Cl. CPC A61B 5/14552; A61B 5/0205; A61B 5/72; A61B 5/02444; A61B 5/0004; G01J 3/12; A61B 5/742

(57) ABSTRACT

This disclosure relates to a pulse oximeter. The pulse oximeter includes a light source emitting detecting light; a light acquirer spaced apart from the light source and so that the detecting light can get the light acquirer after passing through the object; a processor connected to the light acquirer; and a filter on the light path from the light source to the light acquirer. The filter includes a double band filter having center wavelengths of 660 nm and 940 nm. The double band filter can filter the unnecessary light and improve the accuracy of the pulse oximeter.

### CROSS-REFERENCE TO RELATED APPLICATIONS

This application claims all benefits accruing under 35 U.S.C. § 119 from Taiwan Patent Application No. 105141480, filed on Dec. 14, 2016, in the Taiwan Intellectual Property Office, the contents of which are hereby incorporated by reference.

### BACKGROUND

### 1. Technical Field

The present disclosure relates to pulse oximeter.

### 2. Description of Related Art

Pulse oximetry is a noninvasive method for monitoring a person's oxygen saturation. Two light beams are used to radiate the patient's body, usually a fingertip or earlobe, and would be respectively absorbed by the HbO₂ and Hb of the blood. The blood oxygenation can be obtained by processing the intensity change of the two light beams. The light beams with wavelength of 660 nm and 940 nm are preferable for monitoring the oxygen saturation.

However, the light source usually emits a light with continuous wavelength that causes a lots of noise light signal and affects the accuracy of the pulse oximeter. Thus, the light beam is split by a light splitter to two incident light beams with each having continuous wavelength respectively by two filters. Then the two incident light beams with each having continuous wavelength are simultaneously filtered by two filter to form two incident light with wavelength of 660 nm and 940 nm. The two incident light with wavelength of 660 nm and 940 nm are respectively collected by two light acquirers. Thus, the pulse oximeter is complicated and has a lower accuracy.

What is needed, therefore, is to provide a pulse oximeter that can overcome the problems as discussed above.

### BRIEF DESCRIPTION OF THE DRAWINGS

Many aspects of the exemplary embodiments can be better understood with reference to the following drawings. The components in the drawings are not necessarily drawn to scale, the emphasis instead being placed upon clearly illustrating the principles of the exemplary embodiments. Moreover, in the drawings, like reference numerals designate corresponding parts throughout the several views.

FIG. 1 is a schematic section view of a first exemplary embodiment of a pulse oximeter.

FIG. 2 shows a test result of a double band filter of the first exemplary embodiment of the pulse oximeter.

FIG. 3 is a schematic section view of a second exemplary embodiment of a pulse oximeter.

FIG. 4 is a schematic section view of a third exemplary embodiment of a pulse oximeter.

FIG. 5 is a flow chart of the third exemplary embodiment of a method of the pulse oximeter of FIG. 4.

FIG. 6 is a schematic section view of a fourth exemplary embodiment of a pulse oximeter.

FIG. 7 is a flow chart of the fourth exemplary embodiment of a method of the pulse oximeter of FIG. 6.

FIG. 8 is a schematic section view of a fifth exemplary embodiment of a pulse oximeter.

FIG. 9 is a flow chart of the fourth exemplary embodiment of a method of the pulse oximeter of FIG. 8.

FIG. 10 is a flow chart of the fourth exemplary embodiment of another method of the pulse oximeter of FIG. 8.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein can be practiced without these specific details. In other instances, methods, procedures, and components have not been described in detail so as not to obscure the related relevant feature being described. The drawings are not necessarily to scale, and the proportions of certain parts may be exaggerated better illustrate details and features. The description is not to considered as limiting the scope of the exemplary embodiments described herein.

Several definitions that apply throughout this disclosure will now be presented. The term “coupled” is defined as connected, whether directly or indirectly through intervening components, and is not necessarily limited to physical connections. The connection can be such that the objects are permanently connected or releasably connected. The term “outside” refers to a region that is beyond the outermost confines of a physical object. The term “inside” indicates that at least a portion of a region is partially contained within a boundary formed by the object. The term “substantially” is defined to essentially conforming to the particular dimension, shape or other word that substantially modifies, such that the component need not be exact. For example, substantially cylindrical means that the object resembles a cylinder, but can have one or more deviations from a true cylinder. The term “comprising” means “including, but not necessarily limited to”; it specifically indicates open-ended inclusion or membership in a so-described combination, group, series and the like. It should be noted that references to “an” or “one” exemplary embodiment in this disclosure are not necessarily to the same exemplary embodiment, and such references mean at least one.

References will now be made to the drawings to describe, in detail, various exemplary embodiments of the present pulse oximeters.

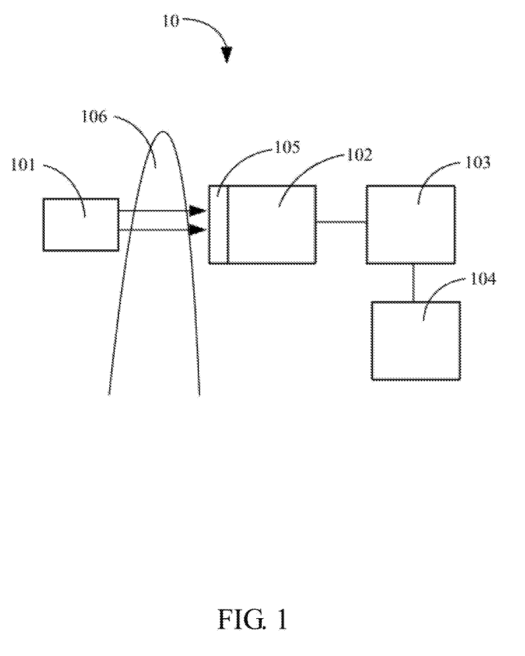

Referring to FIG. 1, a pulse oximeter 10 of a first exemplary embodiment includes a light source 101, a light acquirer 102, a processor 103, a display 104, and a filter 105. In use of the pulse oximeter 10, an object 106, such as a finger, can be located between the light source 101 and the light acquirer 102.

The light source 101 emits detecting light. The light acquirer 102 is spaced apart from the light source 101 so that the detecting light can reach the light acquirer 102 after passing through the object 106. The processor 103 is electrically connected to the light acquirer 102 and the display 104. The acquirer 102 collects and converts the detecting light signal to electric signal, and send the electric signal to the processor 103. The processor 103 obtains a blood oxygenation by processing the electric signal and send the blood oxygenation to the display 104. The display 104 shows the blood oxygenation to the user. The filter 105 is in the light path from the light source 101 to the light acquirer 102 and filters the unnecessary noise light signal and improve the accuracy of the pulse oximeter 10.

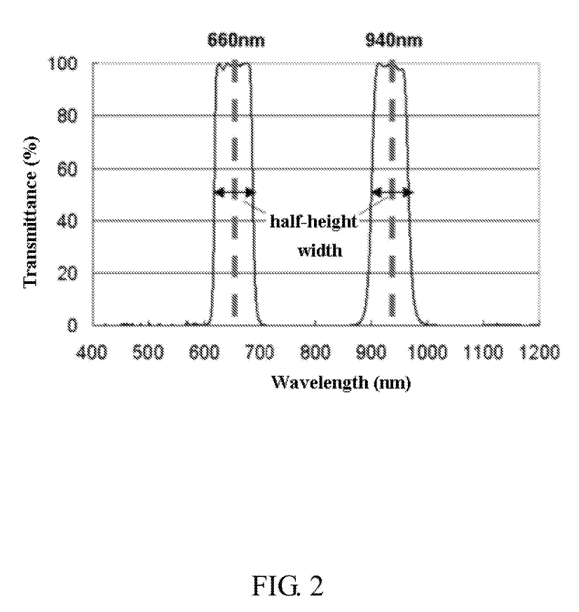

In one exemplary embodiment, the filter 105 is located between the light source 101 to the light acquirer 102. As shown in FIG. 2, the filter 105 includes a double band filter having center wavelength of 660 nm and 940 nm, and a half-height width of 10 nm˜100 nm. The double band filter is a single sheet of light filter that only allows the light with a wavelength of 610 nm˜710 nm and 890 nm˜990 nm to pass through. In one exemplary embodiment, the double band filter only allows the light with a wavelength of 650 nm˜670 nm and 930 nm˜950 nm to pass through. The size and structure of the double band filter is not limited and can be designed according to need.

The filter 105 is adjacent to the light acquirer 102 so that the object 106 can be located between the light source 101 and the filter 105. The filter 105 can be in direct contact with a light receiving surface of the light acquirer 102 or spaced apart from the light acquirer 102.

The structure of the light source 101 is not limited and can be selected according to need. The light source 101 can at least emit light with wavelength of 660 nm and 940 nm. In one exemplary embodiment, the light source 101 is a light emitting diode (LED) and can emit light with wavelength of 400 nm˜1200 nm. The light emitted from the light source 101 is focused on and pass through the object 106. The transmitted light pass through the filter 105 and are substantially and completely collected by the light acquirer 102.

The structure of the light acquirer 102, the processor 103, and the display 104 is not limited and can be selected according to need. The processor 103 can also be connected to a communication unit (not shown) that is used to send the blood oxygenation to other device such as a cell phone, a tablet computer, or a note book. The display 104 is optional.

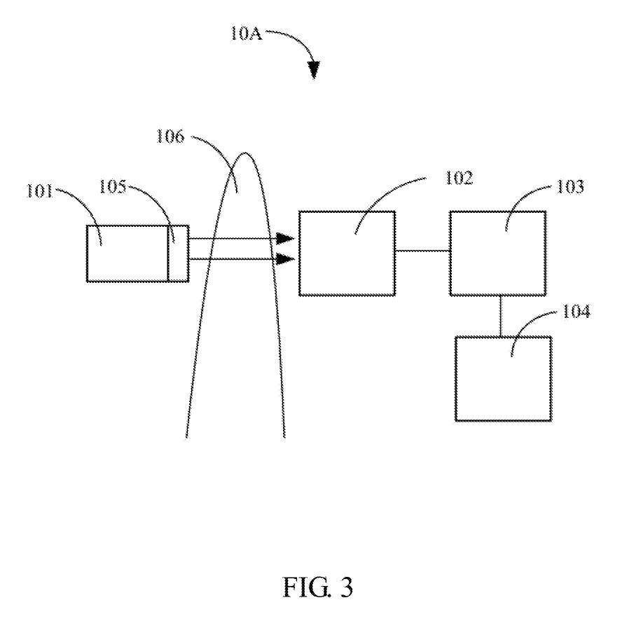

Referring to FIG. 3, a pulse oximeter 10A of a second exemplary embodiment includes a light source 101, a light acquirer 102, a processor 103, a display 104, and a filter 105.

The pulse oximeter 10A of the second exemplary embodiment is similar with the pulse oximeter 10 of the first exemplary embodiment except that the filter 105 is adjacent to the light source 101 so that the object 106 can be located between the filter 105 and the acquirer 102. The light emitted from the light source 101 are filtered by the filter 105 first and then pass through the object 106. Only the light with wavelength of 660 nm and 940 nm can be focused on and pass through the object 106. The transmitted light passed through the object 106 are substantially and completely collected by the light acquirer 102. The filter 105 can be spaced apart from or in direct contact with the light source 101. In one exemplary embodiment, the filter 105 is in direct contact with and fixed on a light exiting surface of the light source 101.

The pulse oximeter 10, 10A has following advantages. First, the filter 105 can filter the unnecessary noise light signal and improve the accuracy of the pulse oximeter 10. Second, because the filter 105 is a double band filter, the pulse oximeter 10 can have only one light source 101 and only one light acquirer 102, and does not need any light splitter. Thus, the pulse oximeter 10 can be more compact and have a lower cost. Third, because the filter 105 is a double band filter, the transmitted light passed through the filter 105 can be substantially completely collected by a single light acquirer 102. Thus, the detected signal has a higher intensity and the pulse oximeter 10 has a higher accuracy.

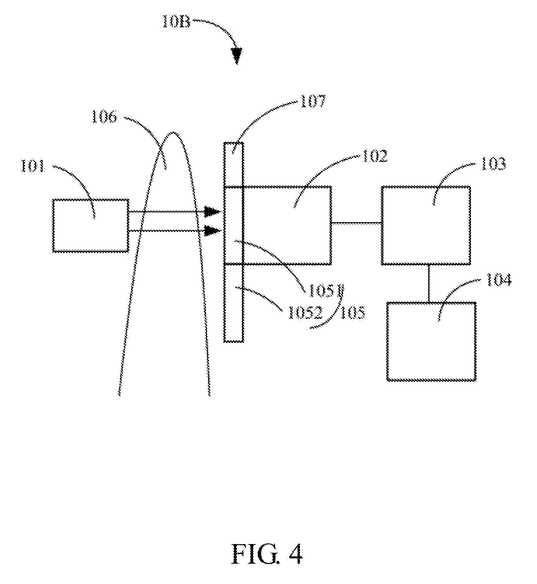

Referring to FIG. 4, a pulse oximeter 10B of a third exemplary embodiment includes a light source 101, a light acquirer 102, a processor 103, a display 104, a filter 105, and a first mechanical device 107.

The pulse oximeter 10B of the third exemplary embodiment is similar with the pulse oximeter 10 of the first exemplary embodiment except that the filter 105 includes a first single band filter 1051 and a second single band filter 1052, and the first mechanical device 107 is provided and connected to the filter 105.

The first single band filter 1051 and the second single band filter 1052 can be arranged side-by-side or stacked with respect to each other. The first single band filter 1051 has a center wavelength of 660 nm and the second single band filter 1052 has a center wavelength of 940 nm. The first mechanical device 107 is used to move the filter 105 so that the first single band filter 1051 and the second single band filter 1052 can be alternately in the light path from the light source 101 to the light acquirer 102.

In one exemplary embodiment, the first single band filter 1051 and the second single band filter 1052 are coplanar and connected to the first mechanical device 107. The first mechanical device 107 can pull or push the filter 105. For example, when the first single band filter 1051 is located on the light acquirer 102, the first mechanical device 107 can make the second single band filter 1052 to be on the light acquirer 102 by pulling; and when the second single band filter 1052 is located on the light acquirer 102, the first mechanical device 107 can make the first single band filter 1051 to be on the light acquirer 102 by pushing.

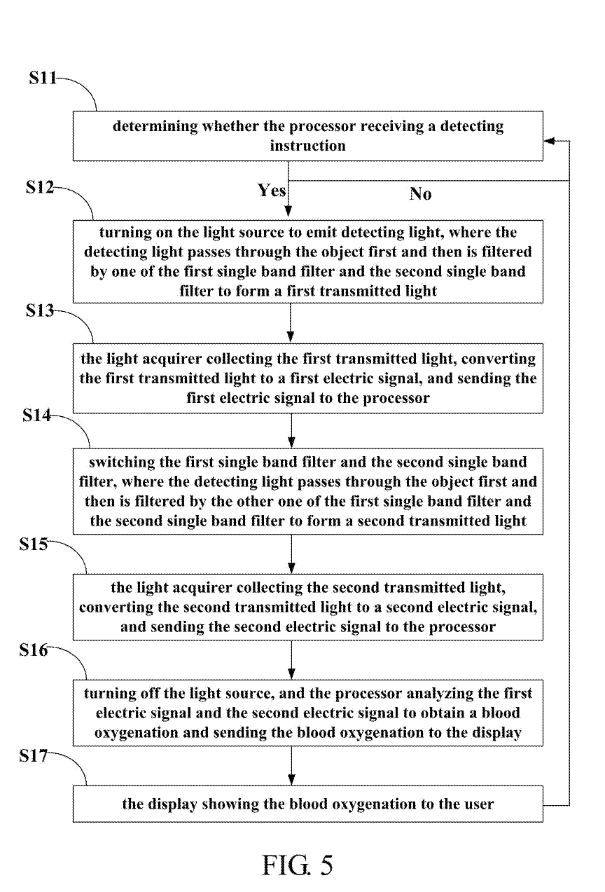

The processor 103 can also control the operation of the pulse oximeter 10B. Referring to FIG. 5, the method of the pulse oximeter 10B includes following steps:

step (S11), determining whether the processor 103 receiving a detecting instruction, if yes, go to step (S12), if no, repeats step (S11);

step (S12), turning on the light source 101 to emit detecting light, where the detecting light passes through the object 106 first and then is filtered by one of the first single band filter 1051 and the second single band filter 1052 to form a first transmitted light, and go to step (S13);

step (S13), the light acquirer 102 collecting the first transmitted light, converting the first transmitted light to a first electric signal, and sending the first electric signal to the processor 103, and go to step (S14);

step (S14), switching the first single band filter 1051 and the second single band filter 1052, where the detecting light passes through the object 106 first and then is filtered by the other one of the first single band filter 1051 and the second single band filter 1052 to form a second transmitted light, and go to step (S15);

step (S15), the light acquirer 102 collecting the second transmitted light, converting the second transmitted light to a second electric signal, and sending the second electric signal to the processor 103, and go to step (S16);

step (S16), turning off the light source 101, and the processor 103 analyzing the first electric signal and the second electric signal to obtain a blood oxygenation and sending the blood oxygenation to the display 104, and go to step (S17); and

step (S17), the display 104 showing the blood oxygenation to the user, and return to step (S11).

In step (S12), the detecting light emitted from the light source 101 pass through the first single band filter 1051 or the second single band filter 1052 since one of the first single band filter 1051 and the second single band filter 1052 is located on the light acquirer 102.

In step (S14), switching the first single band filter 1051 and the second single band filter 1052 is performed by pushing or pulling. Thus, the first single band filter 1051 and the second single band filter 1052 are alternately on the light acquirer 102.

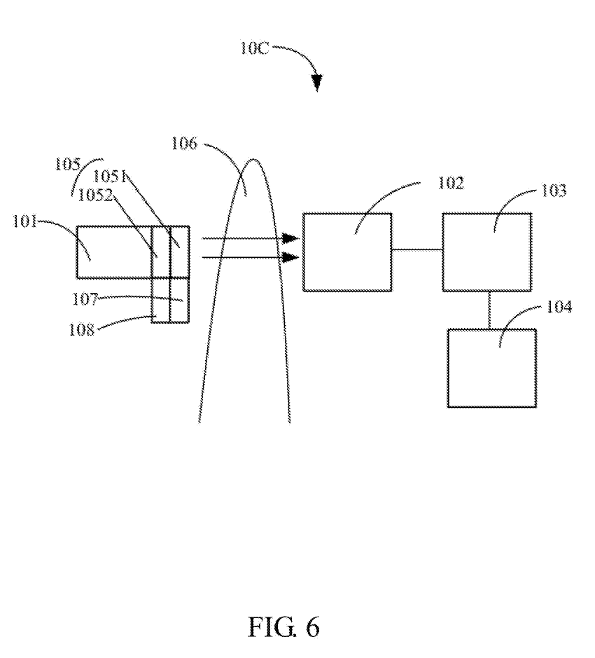

Referring to FIG. 6, a pulse oximeter 10C of a fourth exemplary embodiment includes a light source 101, a light acquirer 102, a processor 103, a display 104, a filter 105, a first mechanical device 107, and a second mechanical device 108.

The pulse oximeter 10C of the fourth exemplary embodiment is similar with the pulse oximeter 10B of the third exemplary embodiment except that the filter 105 in direct contact with a light exiting surface of the light source 101, and the first single band filter 1051 and the second single band filter 1052 are stacked with respect to each other and respectively moved by the first mechanical device 107 and the second mechanical device 108.

In one exemplary embodiment, the first single band filter 1051 is connected to and moved by the first mechanical device 107, and the second single band filter 1052 is connected to and moved by the second mechanical device 108. The first mechanical device 107 can rotate the first single band filter 1051 so that the first single band filter 1051 can be in or moved away from the light path from the light source 101 to the light acquirer 102. The second mechanical device 108 can rotate the second single band filter 1052 so that the second single band filter 1052 can be in or moved away from the light path from the light source 101 to the light acquirer 102. The first single band filter 1051 and the second single band filter 1052 can be located on the light source 101 alternately.

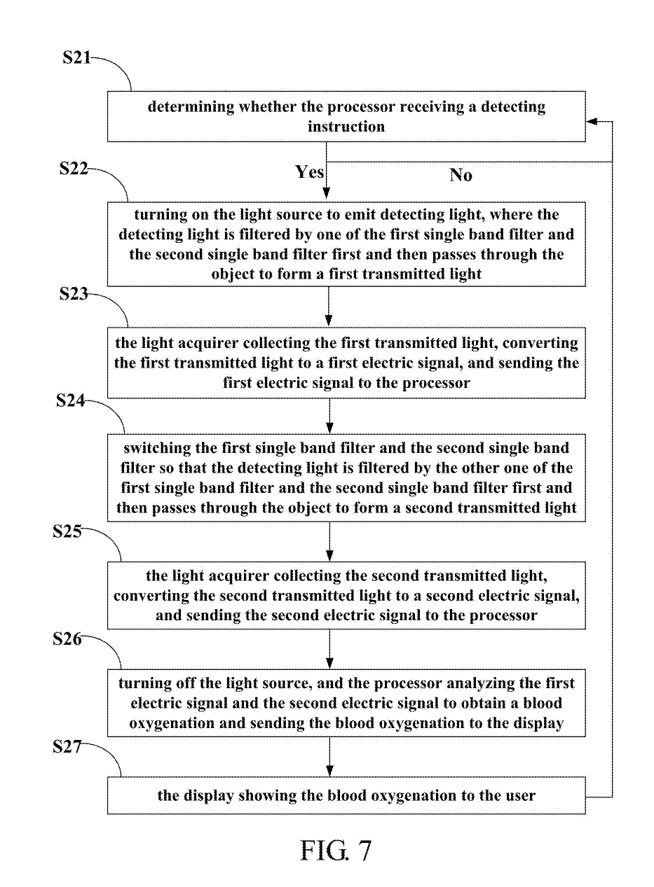

The processor 103 can also control the operation of the pulse oximeter 10C. Referring to FIG. 7, the method of the pulse oximeter 10C includes following steps:

step (S21), determining whether the processor 103 receiving a detecting instruction, if yes, go to step (S22), if no, repeats step (S21);

step (S22), turning on the light source 101 to emit detecting light, where the detecting light is filtered by one of the first single band filter 1051 and the second single band filter 1052 first and then passes through the object 106 to form a first transmitted light, and go to step (S23);

step (S23), the light acquirer 102 collecting the first transmitted light, converting the first transmitted light to a first electric signal, and sending the first electric signal to the processor 103, and go to step (S24);

step (S24), switching the first single band filter 1051 and the second single band filter 1052 so that the detecting light is filtered by the other one of the first single band filter 1051 and the second single band filter 1052 first and then passes through the object 106 to form a second transmitted light, and go to step (S25);

step (S25), the light acquirer 102 collecting the second transmitted light, converting the second transmitted light to a second electric signal, and sending the second electric signal to the processor 103, and go to step (S26);

step (S26), turning off the light source 101, and the processor 103 analyzing the first electric signal and the second electric signal to obtain a blood oxygenation and sending the blood oxygenation to the display 104, and go to step (S27); and step (S27), the display 104 showing the blood oxygenation to the user, and return to step (S21).

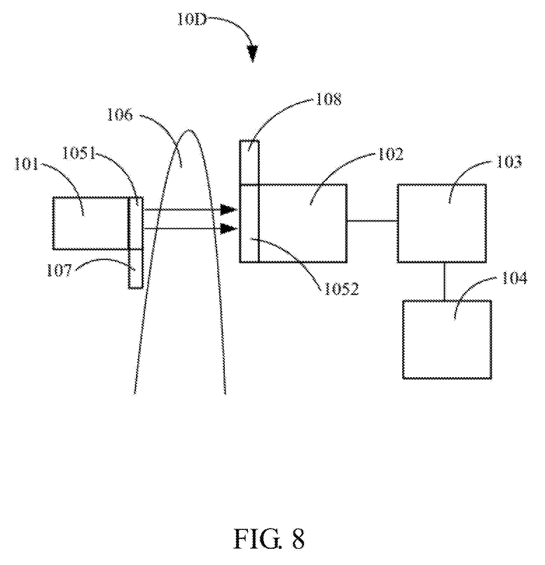

Referring to FIG. 8, a pulse oximeter 10D of a fifth exemplary embodiment includes a light source 101, a light acquirer 102, a processor 103, a display 104, a filter 105, a first mechanical device 107, and a second mechanical device 108.

The pulse oximeter 10D of the fifth exemplary embodiment is similar with the pulse oximeter 10C of the fourth exemplary embodiment except that the first single band filter 1051 in direct contact with a light exiting surface of the light source 101 and moved by the first mechanical device 107, and the second single band filter 1052 in direct contact with the light acquirer 102 and moved by the second mechanical device 108.

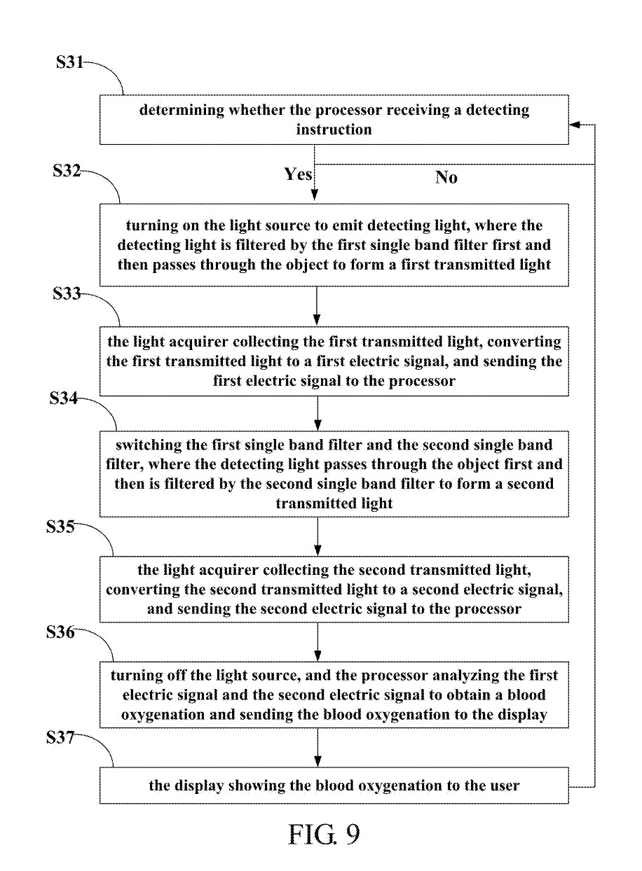

The processor 103 can also control the operation of the pulse oximeter 10D. Referring to FIG. 9, the method of the pulse oximeter 10D includes following steps:

step (S31), determining whether the processor 103 receiving a detecting instruction, if yes, go to step (S32), if no, repeats step (S31);

step (S32), turning on the light source 101 to emit detecting light, where the detecting light is filtered by the first single band filter 1051 first and then passes through the object 106 to form a first transmitted light, and go to step (S33);

step (S33), the light acquirer 102 collecting the first transmitted light, converting the first transmitted light to a first electric signal, and sending the first electric signal to the processor 103, and go to step (S34);

step (S34), switching the first single band filter 1051 and the second single band filter 1052, where the detecting light passes through the object 106 first and then is filtered by the second single band filter 1052 to form a second transmitted light, and go to step (S35);

step (S35), the light acquirer 102 collecting the second transmitted light, converting the second transmitted light to a second electric signal, and sending the second electric signal to the processor 103, and go to step (S36);

step (S36), turning off the light source 101, and the processor 103 analyzing the first electric signal and the second electric signal to obtain a blood oxygenation and sending the blood oxygenation to the display 104, and go to step (S37); and

step (S37), the display 104 showing the blood oxygenation to the user, and return to step (S31).

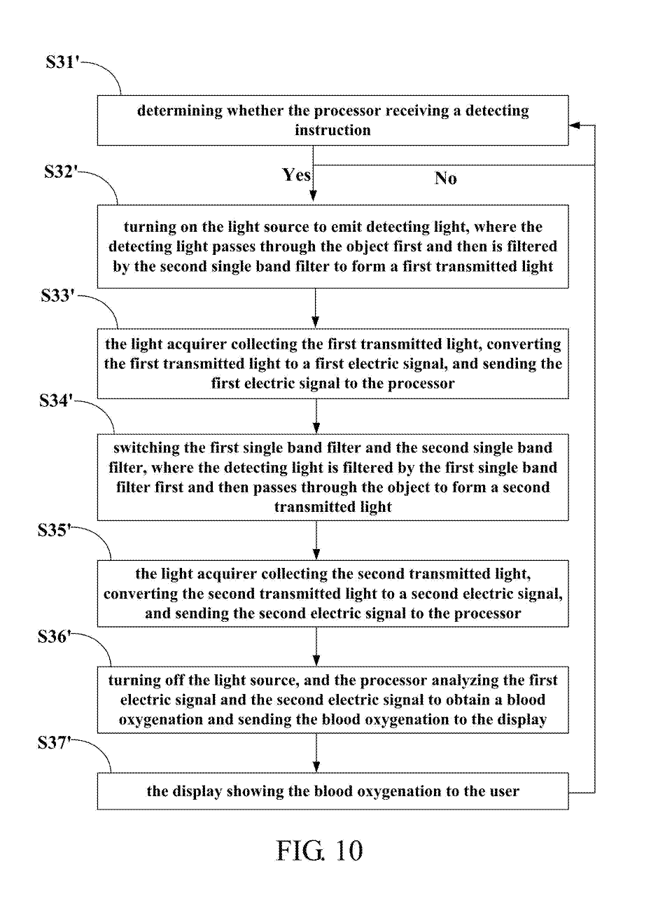

Referring to FIG. 10, the method of the pulse oximeter 10D can also include following steps:

step (S31′), determining whether the processor 103 receiving a detecting instruction, if yes, go to step (S32′), if no, repeats step (S31′);

step (S32′), turning on the light source 101 to emit detecting light, where the detecting light passes through the object 106 first and then is filtered by the second single band filter 1052 to form a first transmitted light, and go to step (S33′);

step (S33′), the light acquirer 102 collecting the first transmitted light, converting the first transmitted light to a first electric signal, and sending the first electric signal to the processor 103, and go to step (S34′);

step (S34′), switching the first single band filter 1051 and the second single band filter 1052, where the detecting light is filtered by the first single band filter 1051 first and then passes through the object 106 to form a second transmitted light, and go to step (S35′);

step (S35′), the light acquirer 102 collecting the second transmitted light, converting the second transmitted light to a second electric signal, and sending the second electric signal to the processor 103, and go to step (S36′);

step (S36′), turning off the light source 101, and the processor 103 analyzing the first electric signal and the second electric signal to obtain a blood oxygenation and sending the blood oxygenation to the display 104, and go to step (S37′); and step (S37′), the display 104 showing the blood oxygenation to the user, and return to step (S31).

The pulse oximeter 10B, 10C, 10D has following advantages. First, the first single band filter 1051 and the second single band filter 1052 can alternately operate and filter the unnecessary noise light signal and improve the accuracy of the pulse oximeter 10. Second, because the first single band filter 1051 and the second single band filter 1052 can alternately operate, the pulse oximeter 10 can have only one light acquirer 102 and only one light source 101, and does not need any light splitter. Thus, the pulse oximeter 10 can be more compact and have a lower cost. Third, because the first single band filter 1051 and the second single band filter 1052 can alternately operate, the transmitted light passed through the filter 105 can be substantially and completely collected by a single light acquirer 102. Thus, the detected signal has a higher intensity and the pulse oximeter 10 has a higher accuracy.

It is to be understood that the above-described exemplary embodiments are intended to illustrate rather than limit the disclosure. Any elements described in accordance with any exemplary embodiments is understood that they can be used in addition or substituted in other exemplary embodiments. Exemplary embodiments can also be used together. Variations may be made to the exemplary embodiments without departing from the spirit of the disclosure. The above-described exemplary embodiments illustrate the scope of the disclosure but do not restrict the scope of the disclosure.

Depending on the exemplary embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

What is claimed is:

1. A pulse oximeter, comprising:
a light source emitting detecting light;
a light acquirer spaced apart from the light source and collecting the detecting light;
a processor connected to the light acquirer; and
a filter on a light path from the light source to the light acquirer, wherein the filter comprises a double band filter having center wavelengths of 660 nm and 940 nm.

2. The pulse oximeter of claim 1, wherein the double band filter has a half-height width of 10 nm-100 nm.

3. The pulse oximeter of claim 1, wherein the double band filter is on the light acquirer so that an object can be between the light source and the double band filter.

4. The pulse oximeter of claim 3, wherein the double band filter is in direct contact with the light acquirer.

5. The pulse oximeter of claim 1, wherein the double band filter is on the light source so that an object can be between the light acquirer and the double band filter.

6. The pulse oximeter of claim 5, wherein the double band filter is in direct contact with the light source.

7. The pulse oximeter of claim 1, further comprising a display electrically connected to the processor.

8. The pulse oximeter of claim 1, further comprising a communication unit electrically connected to the processor.

9. The pulse oximeter of claim 1, wherein the light source comprises a light emitting diode and emits the detecting light with wavelength of 400 nm˜1200 nm.

10. The pulse oximeter of claim 1, wherein the pulse oximeter comprises only one light acquirer, and the filter comprise only one double band filter.
